(19) United States
(12) Patent Application Publication
(10) Pub. No.: US 2017/0296351 A1
(43) Pub. Date: Oct. 19, 2017

(54) VERTEBRAL CAGE ASSEMBLY

(71) Applicant: Stanus Investments, Inc., Dallas, TX (US)

(72) Inventor: Paul A. VAUGHAN, Dallas, TX (US)

(73) Assignee: Stanus Investments, Inc., Dallas, TX (US)

(21) Appl. No.: 15/486,794

(22) Filed: Apr. 13, 2017

Related U.S. Application Data
(60) Provisional application No. 62/323,095, filed on Apr. 15, 2016.

(51) Int. Cl. A61F 2/44; A61F 2/30

(52) U.S. Cl. CPC A61F 2/447; A61F 2/30771; A61F 2002/30904; A61F 2002/30784; A61F 2002/30579; A61F 2002/30331; A61F 2002/4475; A61F 2002/30772

(57) ABSTRACT

Vertebral cage assembly techniques are disclosed. In some embodiments a vertebral cage assembly may include a front wall that includes a first opening, a top wall that includes at least one top growth opening, and a bottom wall that includes at least one bottom growth opening. The vertebral cage assembly may be expandable in a lateral direction.

### CROSS REFERENCE TO RELATED APPLICATIONS

This patent application claims the benefit of provisional patent application No. 62/323,095, filed on Apr. 15, 2016, which is hereby incorporated by reference herein in its entirety.

### FIELD OF THE DISCLOSURE

The present disclosure relates generally to spinal stabilization and correcting spinal deformities. More specifically, but not by way of limitation, the present disclosure relates to vertebral cage assemblies.

### BACKGROUND OF THE DISCLOSURE

The human spine frequently requires surgery to repair deformities or injuries. Spinal problems may be caused by a trauma to the spine received during an accident, excessive strain or stress on the spine from physical activities, a sedentary lifestyle and poor posture that may place abnormal pressure on the spine, disease, or a variety of other reasons. Spinal fusion is a common surgery intended to alleviate pain caused by these spinal deformities or injuries.

The spinal fusion procedure generally includes removing a disk, packing a bone graft between vertebrae and placing a spinal implant, such as a vertebral cage, to fuse elements of the spine together. Once the disk is removed and the bone graft is placed between the vertebrae, the bone graft will fuse to the vertebrae over a period of several months following the surgery.

Placing a conventional vertebral cage between vertebrae may be challenging for several reasons. As well as the complicated surgical processes that may be involved, vertebral cages may need to be sized correctly so that they properly fit between vertebrae and provide surface area contact for promoting bone growth. Vertebrae sizes may vary, however, in a single patient's spine, as well as in different patients. Therefore, providing surface area contact for differently sized vertebrae may be difficult. Moreover, conventional vertebral cages may be large, and fitting such conventional cages between vertebrae may be challenging due to the size. The large size of such cages may cause damage to surrounding tissue and/or vertebrae themselves during placement.

Thus, a need exists for a vertebral cage assembly that overcomes the disadvantages of prior, conventional vertebral cage assemblies.

### SUMMARY OF THE DISCLOSURE

Vertebral cage assembly techniques are disclosed. In some embodiments a vertebral cage assembly may include a front wall that includes a first opening, a top wall that includes at least one top growth opening, and a bottom wall that includes at least one bottom growth opening. The vertebral cage assembly may be expandable in a lateral direction.

In some embodiments, the first opening may have a lateral dimension and a vertical dimension, and the lateral dimension may be greater than the vertical dimension.

In some embodiments, the first opening may have a lateral dimension and a vertical dimension, and the lateral dimension may be less than the vertical dimension.

In some embodiments, the first opening may be configured to receive an expansion tool.

In some embodiments, the at least one top growth opening may have a first size and a first shape.

In some embodiments, the at least one bottom growth opening may be a second size and a second shape.

In some embodiments, the first size and the second size may be the same, and the first shape and the second shape may be the same.

In some embodiments, the first size and the second size may be different, and the first shape and the second shape may be different.

In some embodiments, the at least one top growth opening may include a plurality of top growth openings, and a first number of the plurality of top growth openings may be a first shape, and a second number of the plurality of top growth openings may be a second shape

In some embodiments, the at least one bottom growth opening may include a plurality of bottom growth openings, and a first number of the plurality of bottom growth openings may be a third shape, and a second number of the plurality of bottom growth openings may be a fourth shape.

In some embodiments, the assembly may be shaped such that its dimensions correspond to a vertebra.

In some embodiments, a vertebral cage assembly may include an outer cage and an inner cage coupled to the outer cage, wherein the outer cage and the inner cage are separable from each other in a lateral direction.

In some embodiments, the outer cage may include a front wall that includes a first opening.

In some embodiments, the first opening may have a lateral dimension and a vertical dimension.

In some embodiments, the first opening may overlap with a second opening.

In some embodiments, the inner cage may include a front wall that includes the second opening.

In some embodiments, the first opening and second opening may be configured to receive an expansion tool.

In some embodiments, the outer cage may be sized such that a portion of the inner cage may be fitted inside the outer cage.

In some embodiments, a first dimension of the inner cage may be less than a corresponding first dimension of the outer cage.

In some embodiments, an outer cage of a vertebral cage assembly may include a front wall that may include a first opening having a lateral dimension and a vertical dimension, a top wall that may include at least one top growth opening having a first size and a first shape, and a bottom wall that may include at least one bottom growth opening having a second size and a second shape.

The present disclosure will now be described in more detail with reference to particular embodiments thereof as shown in the accompanying drawings. While the present disclosure is described below with reference to particular embodiments, it should be understood that the present disclosure is not limited thereto. Those of ordinary skill in the art having access to the teachings herein will recognize additional implementations, modifications, and embodiments, as well as other fields of use, which are within the scope of the present disclosure as described herein, and with respect to which the present disclosure may be of significant utility.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be illustrative only.

FIG. 1A shows an outer cage of a vertebral cage assembly according to embodiments of the present disclosure.

FIG. 1B shows an inner cage of a vertebral cage assembly according to embodiments of the present disclosure.

FIG. 1C shows how an outer cage and inner cage of the present disclosure may be fitted together.

FIG. 1D shows a vertebral cage assembly in an unexpanded state according to embodiments of the present disclosure.

FIG. 2 shows how expansion of a vertebral cage assembly may be provided in according to embodiments of the present disclosure.

FIG. 3 shows a vertebral cage assembly after expansion according to embodiments of the present disclosure.

FIG. 4 shows a top view of a vertebra with an unexpanded according to embodiments of the present disclosure.

FIG. 5 shows a top view of a vertebra with an expanded assembly according to embodiments of the present disclosure.

### DETAILED DESCRIPTION

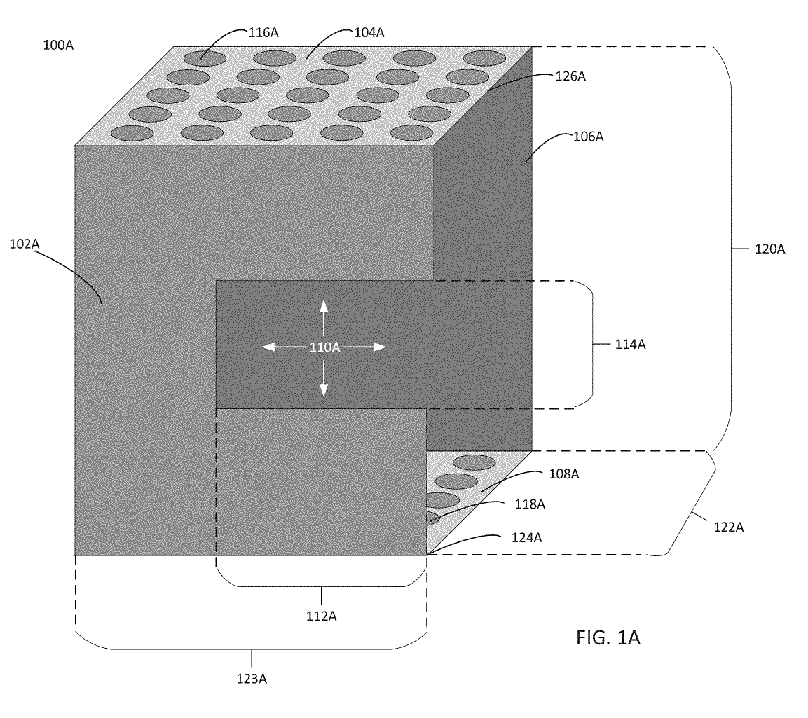

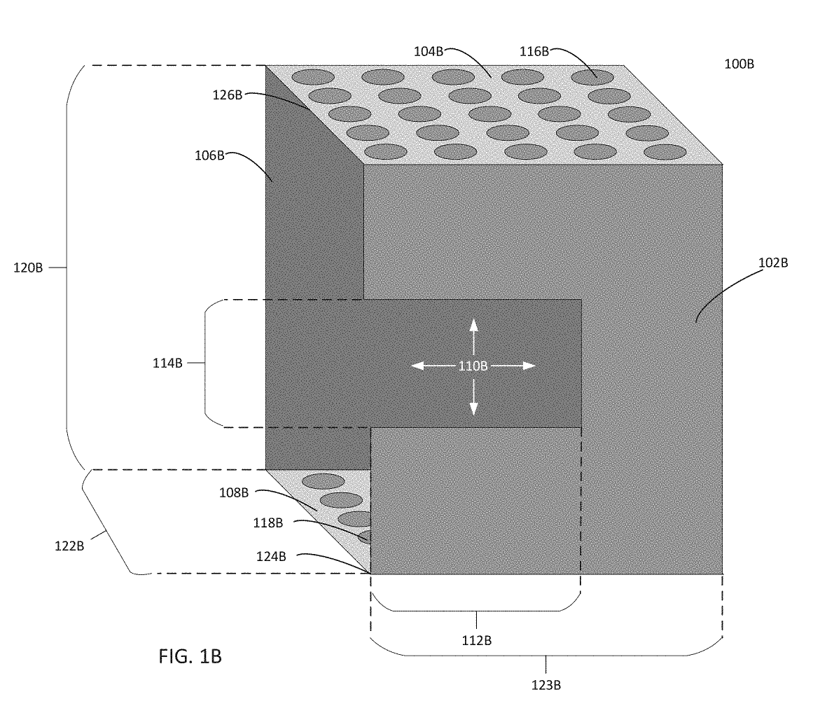

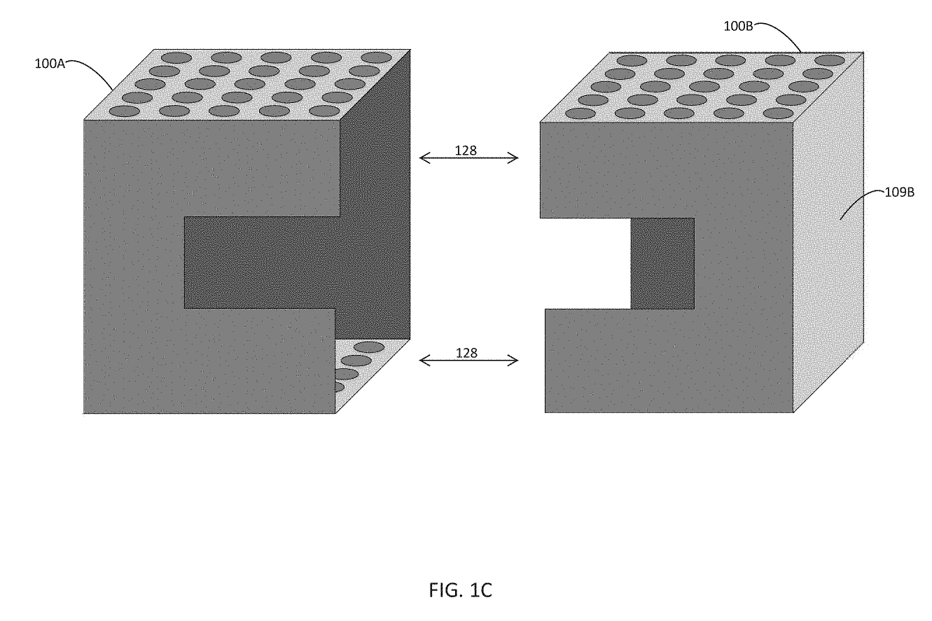

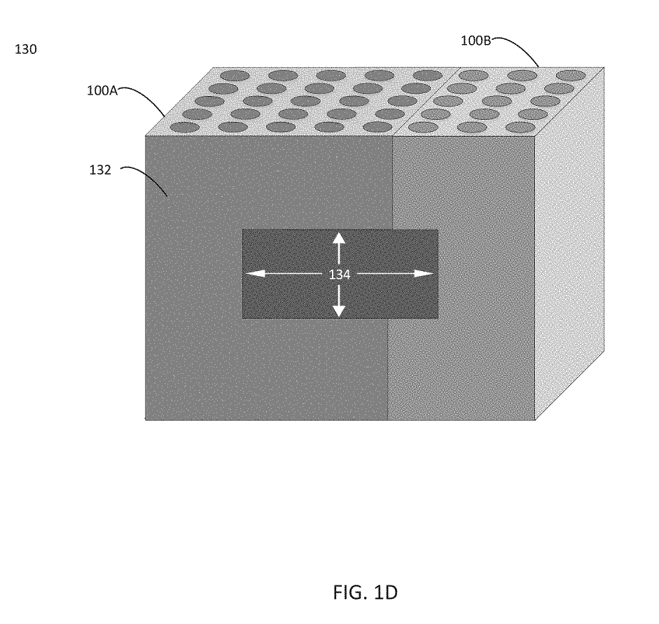

In accordance with embodiments of the present disclosure, a vertebral cage assembly 130 may be placed between vertebrae of a spine. Placement may be provided via an anterior spinal approach, which may advantageously allow for increased visibility in evacuating a spinal disk and reduce the likelihood of nerve damage that may be present in a posterior approach. For example, using an anterior approach may avoid complications that may be associated with a posterior approach, such as dural tears and injury to lumbral sacral nerves. Assembly 130 may be placed between the vertebrae in an unexpanded stated to facilitate easier placement between vertebrae. Assembly 130 may be expanded after placement, and may thereafter promote bone fusion between bone fragments within the vertebral cage assembly and vertebrae above and below the vertebral cage assembly. An outer cage 100A may be fitted with inner cage 100B, as will be explained, to form vertebral cage assembly 130. Vertebral cage assembly 130 in an unexpanded state is shown in FIG. 1D. Outer cage 100A and inner cage 100B are first explained to fully understand how assembly 130 may be formed.

FIG. 1A shows an outer cage 100A of a vertebral cage assembly 130 according to embodiments of the present disclosure. Outer cage 100A may include a front wall 102A, a top wall 104A, a back wall 106A, and a bottom wall 108A. A side wall 109A (not shown in FIG. 1A), may also be included at an end of outer cage 100A that does not receive inner cage 100B.

The front wall 102A may include an opening 110A. Opening 110A may be a predetermined size. The size may be selected based on vertebrae size involved in surgery, an expansion tool requirement, or any number of factors. Opening 110A may have a lateral dimension 112A and a vertical dimension 114A. Lateral dimension 112A is shown as being larger than vertical dimension 114A in FIG. 1A. However, lateral dimension 112A may be smaller than vertical dimension 114A, or the dimensions may be the same size. Opening 110A may alternatively have a different shape to one with dimensions 112A and 114A. For example, opening 110A may have a circular shape, an oval shape, a square shape, a triangular shape, or any other shape as applicable.

Opening 110A may align with an opening 110B of an inner cage 100B (as will be explained below). Opening 110A may accept a tool prong of an expansion tool for expansion of assembly 130. Opening 110A may also allow for insertion of bone fragments within the vertebral cage assembly after the assembly has been placed between vertebrae.

Top wall 104A may have at least one top growth opening 116A. For example, top wall 104A may include a plurality of openings 116A. Each opening 116A may promote bone growth between a vertebra located above top wall 104A and bone fragments disposed internally within the vertebral cage assembly formed by outer cage 100A and inner cage 100B.

Each opening 116A may be circular in shape, as shown in FIG. 1A, or may have a different shape. For example, opening 116A may be square, rectangular, triangular, pentagonal, hexagonal, heptagonal, oxagonal, or star shaped. Opening 116A may be alternatively be any other shape as applicable. If a plurality of openings 116A are provided, a first number of the openings 116A may have a first shape, and a second number of openings 116A may have a second shape. The first and second shapes may be different. Each opening 116A may be sized the same or differently. For example, a first number of openings 116A may have a first size, and a second number of openings 116A may have a second size, where the first and second sizes are different. In FIG. 1A, five rows of openings 116A are shown, where each row includes five openings. However, a different number of rows (more or less than five) may be present in top wall 104A. Moreover, each row may have a different number of openings, the same number of openings, some of the rows may have a different number of openings, and others of the rows may have the same number of openings. Additionally, a plurality of openings 116A on top wall 104A may not be arranged in rows. Rather, for example, the plurality of openings 116A may be scattered on top wall 104A and may not be arranged in rows.

Back wall 106A may be coupled to top wall 104A, bottom wall 108A, and side wall 109A, and may provide structural support to the walls it is coupled to.

Bottom wall 108A may have a number of bottom growth openings 118A. Openings 118A and bottom wall 108A may have the same characteristics as previously discussed regarding openings 116A and top wall 104A. Openings 118A may promote bone growth between a vertebra located below bottom wall 108A and bone fragments disposed internally within the vertebral cage assembly formed by outer cage 100A and inner cage 100B. The number and/or size and/or shape of openings 118A on bottom wall 108A may be the same as the number and/or size and/or shape of openings 116A in top wall 104A. Therefore, top wall 104A with its openings and bottom wall 108A with its openings may be identical or substantially similar parts. Alternatively, the number and/or size and/or shape of openings 118A on bottom wall 108A may be different to the number and/or size and/or shape of openings 116A in top wall 104A.

The distance between top wall 104A and bottom wall 108A is identified as 120A in FIG. 1A. Distance 120A may be sized in relation to an inner cage 100B. The distance between front wall 102A and back wall 106A is identified 122A in FIG. 1A. Distance 122A may be smaller than distance 120A (as shown in FIG. 1A), may be the same or substantially the same as distance 120A, or may be larger than distance 120A. Moreover, distance 120A may be sized in relation to an inner cage 100B. Distance 123A is the distance between sidewall 109A (not shown) and the side of outer cage 100A that receives inner cage 100B.

Side wall 109A may be coupled to front wall 102A, top wall 104A, back wall 106A, and bottom wall 108A, and may provide structural support to the walls it is coupled to.

Walls 102A, 104A, 106A, 108A, and 109A may be formed from a unitary material, thereby providing that outer cage 100A is not made from composite walls that are coupled together. Alternatively, one or more of walls 102A, 104A, 106A, 108A, and 109A may be coupled together with a bonding agent or welding to form outer cage 100A. Additionally, certain walls may be formed from a unitary material, and other of the walls may be coupled to the unitary walls to form outer cage 100A.

Walls 102A, 104A, 106A, 108A, and 109A may be made of a material configured for placement between vertebrae. Each wall may be made from the same material. Alternatively, the walls may be made of different materials when outer cage 100A is formed by coupling together each or some of the walls.

The edges/ends and/or corners of outer cage 100A may be rounded. For example, corner 124A and edge/end 126A may be rounded. Such rounding may provide for improved ease of placement between vertebrae, as well as an improved fit between outer cage 100A and inner cage 100B (as will be discussed). Corner 124A and edge/end 126A are discussed in regard to FIG. 1A as examples, and it should be noted that all other edges/ends and/or corners of outer cage 100A may also be rounded.

Rounding of some or all of edges/ends and/or corners in outer cage 100A (as well as inner cage 100B, and assembly 130 as a whole) may be beneficial when placing assembly 130 within a patient's body because it may reduce tissue damage. Instead of unintentionally damaging tissue surrounding vertebrae, the rounding may avoid catching or severing such tissue.

FIG. 1B shows an inner cage 100B of a vertebral cage assembly according to embodiments of the present disclosure. Elements of inner cage 100B with the same reference numbering as FIG. 1A (excluding the “A” and “B” identifiers) may be as discussed above in regard to FIG. 1A.

Distances 120B and 122B may be sized such that inner cage 100B fits moveably, but snugly within outer cage 100A. For example, distances 120B may be sized such that it is less than, but within a predetermined size range of distance 120A. Instead (or in addition), distance 122B may be sized such that it is less than, but within a predetermined size range of distance 122B. The predetermined size range may be a range that allows for snug placement between inner cage 100B and outer cage 100A.

Like opening 110A, opening 110B may accept a tool prong of an expansion tool for expansion of a assembly 130. Opening 110B may also allow for insertion of bone fragments within the vertebral cage assembly after the assembly has been placed between vertebrae.

As shown by FIGS. 1A and 1B, each of outer cage 100A and inner cage 100B may have a rectangular box shape. However, different shaped outer and inner cages are contemplated by the present disclosure. For example, outer cage 100A and/or inner cage 100B may be shaped like parts of a vertebra in a patient's body. This shaping may provide that assembly 130 is shaped like a vertebra and fits easier and more naturally between vertebrae, and may therefore more comfortable for a patient. Assembly 130 may be shaped like a vertebra by corresponding its dimensions to dimensions of a model vertebra or a vertebra in the spine where assembly 130 is to be placed.

FIG. 1C shows how outer cage 100A and inner cage 100B may be fitted together. Outer cage 100A and inner cage 100B may be moved toward each other as shown by double sided arrows 128, and outer cage 100A may receive inner cage 100B. Inner cage 100B may be fitted snugly within outer cage 100A. The surfaces of outer cage 100A and/or inner cage 100B that contact each other during fitting may be keyed or roughened to aid in a snug fit between the cages. Moreover, one or more surfaces of outer cage 100A and/or inner cage 100B that contact each other during fitting may be ratcheted, and may engage with each other during fitting and expansion. The ratcheting may provide that assembly 130 may not be returned to its unexpanded width after expansion has occurred. FIG. 1C also shows side wall 109B of inner cage 100B.

FIG. 1D shows vertebral cage assembly 130 in an unexpanded state according to embodiments of the present disclosure where inner cage 100B is fitted snugly within outer cage 100A. Inner cage 100B may be pushed farther into outer cage 100A than as shown in FIG. 1D, or alternatively may not be pushed as far into outer cage 100A as shown in FIG. 1D. The fitting may provide that an overall front wall 132 of assembly 130 is formed. When inner cage 100B and outer cage 100A are fitted together, opening 110B may be aligned with opening 110A. For example, openings 110B and 110A may substantially overlap as shown in FIG. 1D such that an overall opening 134 into vertebral cage assembly is provided. Opening 134 may have a lateral dimension that is larger than its vertical dimension. Alternatively, the lateral and vertical dimensions of opening 134 may be the same, or substantially the same, or the vertical dimension may be larger than the lateral dimension.

In operation, bone fragments may be initially paced inside both (or alternatively, one of) outer cage 100A and inner cage 100B. Inserting inner cage 100B into outer cage 100A may form vertebral cage assembly 130 in an unexpanded state. Assembly 130, in its unexpanded state, may then be placed between two vertebrae in a patient's back during a surgical procedure. The reduced size (e.g., reduced width) of assembly 130 in its unexpanded state may allow for easier placement of assembly 130 between vertebrae. Once placed between vertebrae, assembly 130 may be expanded. An expansion tool may be used to expand assembly 130.

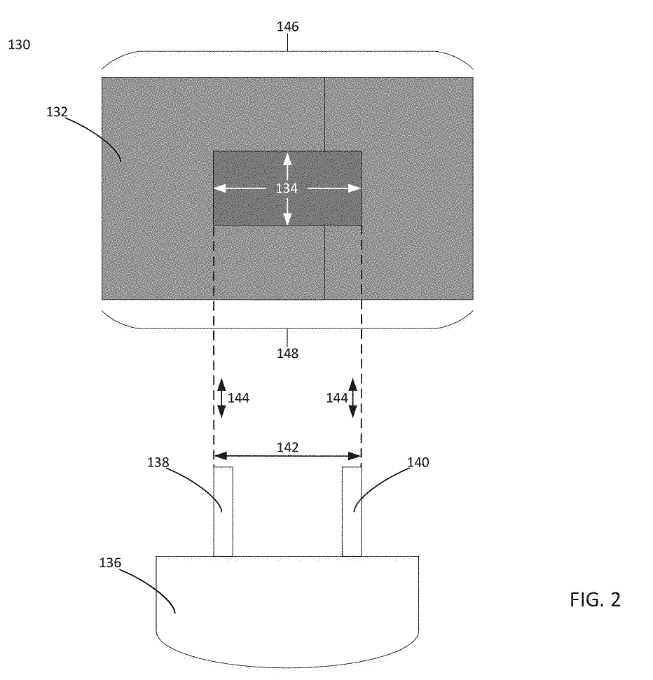

FIG. 2 shows how expansion of assembly 130 may be provided in accordance with embodiments of the present disclosure. As shown in FIG. 2, an expansion tool 136 may be provided to expand assembly 130. Expansion tool 136 may have a first prong 138 and a second prong 140. Prongs 138 and 140 may be initially form a width 142. Width 142 may correspond to the width of opening 134 such that prongs 138 and 140 can be inserted into opening 134. Prongs 138 and 140 may be inserted into opening 134 by bringing tool 136 and assembly 130 together as indicated by double sided arrows 144. Before expansion the top surface of assembly 130 may have a width equaling distance 146 as shown by FIG. 2. Moreover, a bottom surface of assembly may have a width equaling a distance 148 as shown in FIG. 2.

Expansion of assembly 130 may be provided by laterally moving prongs 138 and 140 away from each other when the prongs are inserted into assembly 130. This movement causes contact between one or more of prongs 138 and 140, and outer cage 100A and/or inner cage 100B. The contact causes movement of inner cage 100B and outer cage 100A laterally away from each other, and expands assembly 130. This expansion may provide that a top surface of assembly 130 (formed by top wall 104A and top wall 104B) and a bottom surface of assembly 130 (formed by bottom wall 108A and bottom wall 108B) have greater surface area contact with adjacent vertebrae that are located above and/or below assembly 130. The greater surface area contact may increase the amount of possible bone growth between the adjacent vertebrae and bone fragments disposed internally within assembly 130.

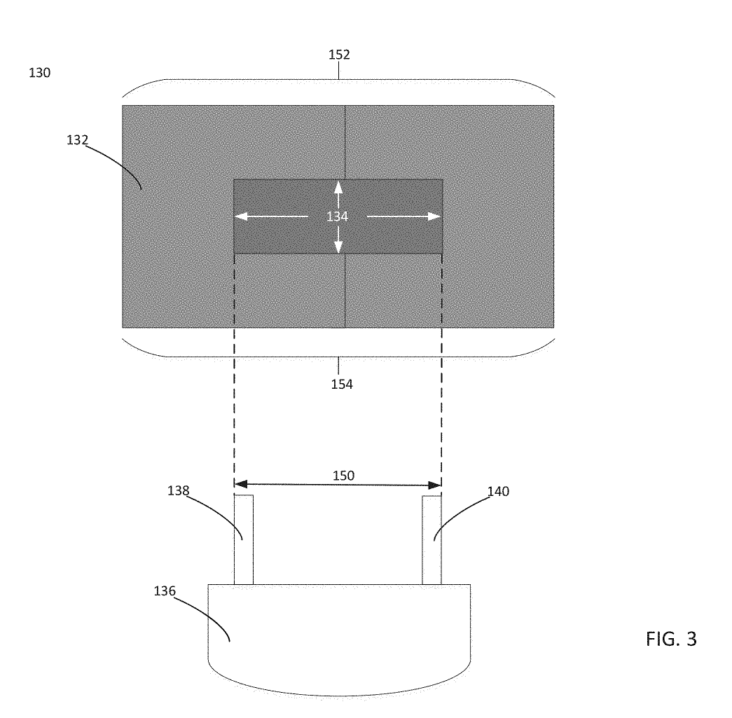

FIG. 3 shows assembly 130 after expansion has occurred to a desired width and tool 136 has been removed. First prong 138 and second prong 140 of tool 136 now form width 150 due to the movement of prongs 138 and 140 away from each other. Width 150 may be larger than width 142. Additionally the top surface of assembly 130 may now have a width that equals a distance 152 as shown by FIG. 3. A bottom surface of assembly may now have a width that equals a distance 154. Distances 152 and 154 of expanded assembly 130 may be larger that distances 146 and 148 of unexpanded assembly 130. As shown by FIG. 3, assembly 130 now has an increased surface area on its top and bottom surfaces, which may increase the amount of possible bone growth between adjacent vertebrae and bone fragments disposed internally within assembly 130.

Assembly 130 may be expanded a predetermined threshold amount that allows assembly 130 to maintain structural integrity, and prevent inner cage 100B from decoupling from outer cage 100A. Once assembly 130 has been expanded to a desired width, additional bone fragments may be introduced into assembly 130 via opening 134. Bone growth between adjacent vertebrae and the bone fragments disposed internally within assembly 130 may then occur.

Tool 136 may operate by moving both of prongs 138 and 140 away from each other to expand assembly 130. Alternatively, one of prongs 138 and 140 may be fixed, and the other of prongs 138 and 140 may be configured to move away from the fixed prong.

As discussed above, one or more surfaces of outer cage 100A and/or inner cage 100B that contact each other during fitting may be ratcheted, and may engage with each other during fitting and expansion. The ratcheting may provide that assembly 130 may not be returned to its unexpanded width after expansion has occurred. Furthermore, one or more surfaces of outer cage 100A and/or inner cage 100B that contact adjacent vertebrae (e.g., top and/or bottom walls of assembly 130) may include serrations. The serrations may couple outer cage 100A and/or inner cage 100B to the adjacent vertebrae and provide that assembly 130 is locked in its expanded state and does not return to its unexpanded width.

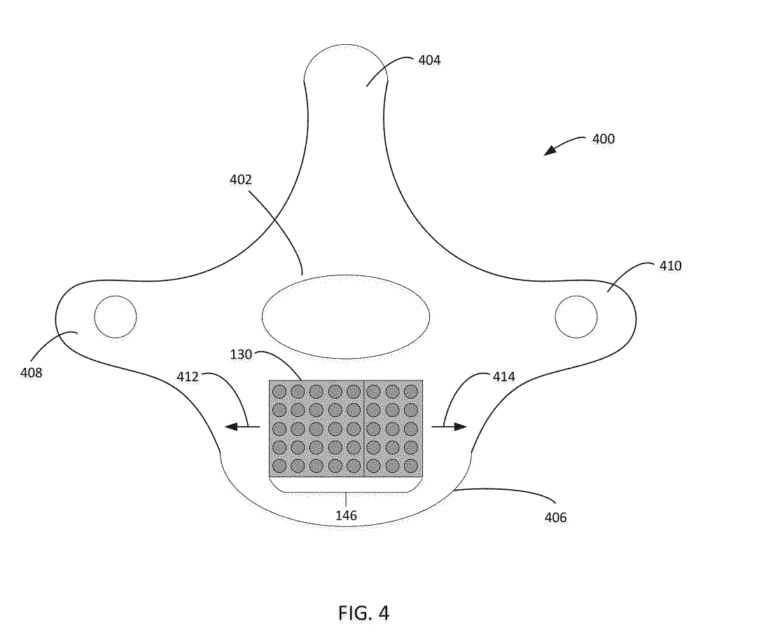

FIG. 4 shows a top view of a vertebra 400, such as a cervical, a lumbar, or a thoracic vertebra, with an unexpanded assembly 130. Vertebra 400 includes pedicle 402, a posterior side of vertebra 404, and an anterior side 406 of vertebra 400. Vertebra 400 further includes a left side 408 and a right side 410. As shown by FIG. 4, unexpanded assembly 130 may be located proximate to anterior side 405 of vertebra 400. However, assembly 130 may alternatively be located in other areas of vertebra 400. The view from FIG. 4 shows the top surface of unexpanded assembly 130. The top surface has a width that equals distance 146, as previously discussed.

As shown by FIG. 4, assembly 130 may be expanded in the directions shown by arrows 412 and 414. For example, outer cage 100A may move in the direction shown by arrow 412 during expansion, and inner cage 100B may move in the direction shown by arrow 414 during expansion.

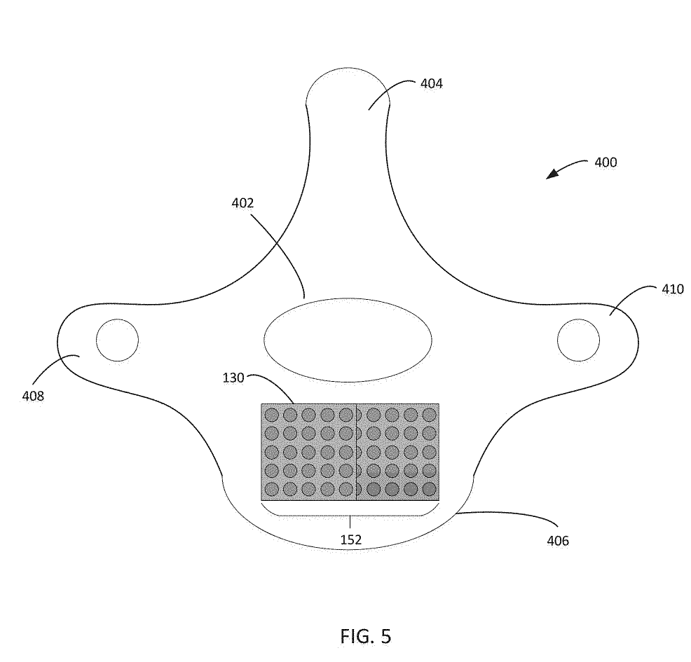

FIG. 5 shows a top view of a vertebra 400 with an expanded assembly 130. As shown by FIG. 5, assembly 130 is in its expanded state and its top surface has a width that equals distance 152. Therefore, assembly 130 has an expanded surface contact area which may promote bone growth as discussed.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of at least one particular implementation in at least one particular environment for at least one particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. A vertebral cage assembly, comprising:
a front wall that includes a first opening;
a top wall that includes at least one top growth opening; and
a bottom wall that includes at least one bottom growth opening;
wherein the vertebral cage assembly is expandable in a lateral direction.

2. The assembly of claim 1, wherein the first opening has a lateral dimension and a vertical dimension, and the lateral dimension is greater than the vertical dimension.

3. The assembly of claim 1, wherein the first opening has a lateral dimension and a vertical dimension, and the lateral dimension is less than the vertical dimension.

4. The assembly of claim 1, wherein the first opening is configured to receive an expansion tool.

5. The assembly of claim 1, wherein the at least one top growth opening is a first size and a first shape.

6. The assembly of claim 5, wherein the at least one bottom growth opening is a second size and a second shape.

7. The assembly of claim 6, wherein the first size and the second size are the same, and the first shape and the second shape are the same.

8. The assembly of claim 6, wherein the first size and the second size are different, and the first shape and the second shape are different.

9. The assembly of claim 1, wherein the at least one top growth opening includes a plurality of top growth openings, and a first number of the plurality of top growth openings are a first shape, and a second number of the plurality of top growth openings are a second shape

10. The assembly of claim 9, wherein the at least one bottom growth opening includes a plurality of bottom growth openings, and a first number of the plurality of bottom growth openings are a third shape, and a second number of the plurality of bottom growth openings are a fourth shape.

11. The assembly of claim 1, wherein the assembly is shaped such that its dimensions correspond to a vertebra.

12. A vertebral cage assembly comprising:
an outer cage; and
an inner cage coupled to the outer cage;
wherein the outer cage and the inner cage are separable from each other in a lateral direction.

13. The vertebral cage assembly of claim 12, wherein the outer cage comprises a front wall that includes a first opening.

14. The vertebral cage assembly of claim 13, wherein the first opening has a lateral dimension and a vertical dimension.

15. The vertebral cage assembly of claim 14, wherein the first opening overlaps with a second opening

16. The vertebral cage assembly of claim 15, wherein the inner cage includes a front wall that includes the second opening.

17. The vertebral cage assembly of claim 16, wherein the first opening and second opening are configured to receive an expansion tool.

18. The vertebral cage assembly of claim 12, wherein the outer cage is sized such that a portion of the inner cage is fitted inside the outer cage.

19. The vertebral cage assembly of claim 12, wherein a first dimension of the inner cage is less than a corresponding first dimension of the outer cage.

20. An outer cage of a vertebral cage assembly, comprising:
a front wall that includes a first opening having a lateral dimension and a vertical dimension;
a top wall that includes at least one top growth opening having a first size and a first shape; and
a bottom wall that includes at least one bottom growth opening having a second size and a second shape.
